SEQUENCE LISTING (20 sequences)

SEQ ID NO: 1 - DNA, 15 bp, Homo sapiens
ccagcattatgaaag

SEQ ID NO: 2 - DNA, 40 bp, Homo sapiens
gtaagtctgccagcattatgaaagtgaatcttacttttgt

SEQ ID NO: 3 - DNA, 32 bp, Homo sapiens
gtaagtctgccagcattatgaaagtgaatctt

SEQ ID NO: 4 - DNA, 20 bp, Artificial Sequence
  Synthetic
gcattatgaaagtgaatctt

SEQ ID NO: 5 - DNA, 21 bp, Artificial Sequence
  Synthetic
gtaagtctgaaagtgaatctt

SEQ ID NO: 6 - DNA, 33 bp, Artificial Sequence
  Synthetic
gtaagtctgccagcaattatgaaagtgaatctt

SEQ ID NO: 7 - DNA, 21 bp, Artificial Sequence
  Synthetic
gtaagtctgccagtgaatctt

SEQ ID NO: 8 - DNA, 21 bp, Artificial Sequence
  Synthetic
gtaagtctgccagtgaatctt

SEQ ID NO: 9 - DNA, 19 bp, Artificial Sequence
  Synthetic
gtaagtctgccagcatctt

SEQ ID NO: 10 - DNA, 18 bp, Artificial Sequence
  Synthetic
gtaagtctgccagctctt

SEQ ID NO: 11 - DNA, 33 bp, Artificial Sequence
  Synthetic
gtaagtctgccagcatttatgaaagtgaatctt

SEQ ID NO: 12 - DNA, 29 bp, Artificial Sequence
  Synthetic
gtaagtctgccattatgaaagtgaatctt

SEQ ID NO: 13 - DNA, 18 bp, Artificial Sequence
  Synthetic
gtaagtctgccagctctt

SEQ ID NO: 14 - DNA, 23 bp, Artificial Sequence
  Synthetic
aagattcactttcataatgctgg

SEQ ID NO: 15 - DNA, 26 bp, Artificial Sequence
  Synthetic
agtctgccagcattatgaaagtgaat

SEQ ID NO: 16 - DNA, 72 bp, Homo sapiens
aattaaggagtaagtctgccagcattatgaaagtgaatcttacttttgtaaaactttatggtttgtggaaaa

SEQ ID NO: 17 - DNA, 22 bp, Artificial Sequence
  Synthetic
aattaaggagtaagtctgccag

SEQ ID NO: 18 - DNA, 22 bp, Artificial Sequence
  Synthetic
cataaagttttacaaaagtaag

SEQ ID NO: 19 - DNA, 26 bp, Artificial Sequence
  Synthetic
ttctcttgatgatgctgatgctttgg

SEQ ID NO: 20 - DNA, 26 bp, Artificial Sequence
  Synthetic
gtctgatcgtttctttagtggtgtca

(19) United States
(12) Patent Application Publication
(10) Pub. No.: US 2021/0030851 A1
(43) Pub. Date: Feb. 4, 2021

(54) COMPOSITIONS AND METHODS FOR TREATING SPINAL MUSCULAR ATROPHY

(71) Applicant: APPLIED STEMCELL, INC., Milpitas, CA (US)

(72) Inventors: Ling-jie KONG, Union City, CA (US); Ruby Yanru TSAI, San Jose, CA (US); Zoya GLUZMAN-POLTORAK, Livermore, CA (US); Ivka AFRIKANOVA, San Jose, CA (US)

(21) Appl. No.: 17/072,056

(22) Filed: Oct. 16, 2020

Related U.S. Application Data
(63) Continuation-in-part of application No. PCT/US2019/027775, filed on Apr. 17, 2019.
(60) Provisional application No. 62/659,119, filed on Apr. 17, 2018.

(51) Int. Cl. A61K 38/46; C12N 15/113; A61K 9/00

(52) U.S. Cl. CPC A61K 38/465; A61K 9/0019; C12N 15/113

(57) ABSTRACT

Provided herein are compositions and methods for enhancing expression of SMN protein in a cell. In one embodiment, the composition comprises a site-specific nuclease targeting the ISS-N1 region of human SMN2 gene. Also provided are compositions and methods for treating or ameliorating spinal muscular atrophy.

### CROSS-REFERENCE TO RELATED APPLICATIONS

The present application is a continuation-in-part application of PCT application no. PCT/US2019/027775, filed Apr. 17, 219, which claims the benefit of U.S. provisional patent application 62/659,119, filed Apr. 17, 2018, the disclosure of which is incorporated herein by reference in the entirety.

### FIELD OF THE INVENTION

The present invention generally relates to compositions and methods for treating or ameliorating spinal muscular atrophy.

### BACKGROUND

Spinal muscular atrophy (SMA) is the leading genetic cause of infantile death. SMA is characterized by the degeneration of alpha-motor neurons in the spinal cord, leading to progressive muscle weakness followed by respiratory insufficiency. SMA is mainly caused by low levels of Survival Motor Neuron (SMN) protein due to homozygous deletion or mutational of the SMN1 gene. In humans, SMN protein is also encoded by SMN2 gene. However, SMN2 gene, as compared to the SMN1 gene, possesses a point mutation within exon 7, leading to an altered splicing in most SMN2 mRNA that lacks exon 7. The resultant truncated SMN protein is unstable and hypofunctional. Normally, SMN1 produces abundant SMN proteins. However, homozygous mutation of SMN1 results in only a small amount of functional SMN protein produced by SMN2 gene, leading to SMA.

Several approaches have been explored to treat or ameliorate SMA. :For example, antisense oligonucleotide has been used to modify pre-messenger RNA splicing of the SMN2 gene and thus promotes production of full-length SMN protein (see Finkel R S et al., Nusinersen versus Sham Control in Infantile-Onset Spinal Muscular Atrophy, N Engl J Med (2017) 377:1723-32; U.S. Pat. No 7,838,657). Functional replacement of the mutated SMN1 gene has also been tested and showed efficacy of this gene therapy (Mendell J R et al., Single-Dose Gene-Replacement Therapy for Spinal Muscular Atrophy, N Engl J Med (2017) 377:1713-22). Notably, antisense oligonucleotide-mediated splicing correction of SMA was recently approved by US Food and Drug Administration (FDA), However, the approved treatment needs 6 spinal injections each year, which costs $625,000 to $759,000 in the first year and about $375,000 annually afterwards. In addition, the antisense oligonucleotide-mediated treatment also has many side effects. Therefore, there is a continuing need to develop new treatment for SMA.

### SUMMARY OF THE INVENTION

In one aspect, the present disclosure provides a composition that is capable of enhancing expression of SMN protein in a cell. In certain embodiments, the composition includes a site-specific nuclease that targets the ISS-N1 region of human SMN2 gene. In some embodiments, the site-specific nuclease is a CRISPR-associated (Cas) nuclease, a zinc finger nuclease or a TALEN nuclease.

In one embodiment, the composition comprises (1) a CRISPR-associated (Cas) nuclease or a nucleic acid encoding the same; and (2) a gRNA or a nucleic acid encoding the same, wherein the gRNA targets the ISS-N1 region of human SMN2 gene.

In certain embodiments, the composition described herein comprises a nucleic acid encoding the Cas nuclease or the gRNA, wherein the nucleic acid is contained in a viral vector.

In certain embodiments of the composition described herein, the ISS-N1 region comprises SEQ ID NO: 1 (CCAGCATTATGAAAG). In some embodiments, the ISS-N1 region consists of SEQ ID NO: 1.

In some embodiments, the composition described herein is capable of increasing inclusion of exon 7 of SMN2 mRNA in the cell.

In some embodiments, the composition described herein is capable of generating a modified ISS-N1 in the cell when the composition is introduced to the subject. In some embodiments, the composition further comprises a second gRNA or a nucleic acid encoding the same, wherein the second gRNA targets the modified ISS-N1.

In certain embodiments, the composition described herein is capable of ameliorating at least one symptom of SMA when administered into a subject having SMA.

In another aspect, the present disclosure provides a method of enhancing expression of SMN protein in a human cell. In one embodiment, the method comprises introducing the composition described herein to the human cell.

In certain embodiments, the cell is a motor neuron.

In yet another aspect, the present disclosure provides a method for treating or ameliorating spinal muscular atrophy (SMA). In one embodiment, the method comprises administering a therapeutically effective amount of the composition described herein to a subject having at least one symptom associated with spinal muscular atrophy (SMA).

In certain embodiments of the method described herein, the subject is an infant.

In certain embodiments of the method described herein, the composition is administered systematically to the subject. In certain embodiments, the composition is administrated through subcutaneous injection. In certain embodiments, the composition is administrated through intravenous injection.

In certain embodiments of the method described herein, the composition is administered into the central nervous system of the subject. In certain embodiments, the composition is administered into the cerebrospinal fluid of the subject. In certain embodiments, the composition is administered into the intrathecal space of the subject.

In certain embodiments, the method described herein ameliorates at least one symptom of SMA in the subject. In some embodiments, inclusion of exon 7 of SMN2 mRNA in a motor neuron in the subject is increased.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the exon6-exon8 region of human SMN2 gene. The sequence of the 5′ end of the intron 7 region is illustrated with the ISS-N1 region sequence underlined.

FIGS. 2A and 2B show that CRISPR targeting with a gRNA leads to increased SMN expression. SMN1 knock-out cell line (SMN1 −/−) was generate. spCAS9/gRNA RNP complex was used for transfection. The gRNA targeting sequence was underlined and the CAS9 cutting site was indicated by an arrow head in the wild type sequence (Ctrl). Single cell clones were generated and genotyped by sequencing. The genomic sequence for each homozygous clone were shown (FIG. 2A). FIG. 2B shows the RT-PCR results performed with mRNA from each clone with primers SMNex6-F and Exon8 Primer R. The top band shown indicated the splicing form includes exon7 (ex678) and the bottom band indicates the one with Exon7 skipping (ex6_8).

FIG. 3 shows an illustrative gRNA targeting the ISS-N1 region using spCas9. The nucleotides with underline in SEQ ID NO: 2 represent the ISS-N1 region. The horizonal line with arrow represents the region being targeted by the gRNA. The vertical arrow shows the position of double strand break being introduced.

FIG. 4 shows an illustrative gRNA targeting the ISS-N1 region using saCas9. The nucleotides with underline in SEQ ID NO: 2 represent the ISS-N1 region. The horizonal line with arrow represents the region being targeted by the gRNA. The vertical arrow shows the position of double strand break being introduced.

FIG. 5 illustrates the deletion of the ISS-N1 region using two gRNAs and xCas9-3.7. The nucleotides with underline in SEQ ID NO: 16 represent the ISS-N1region. The horizonal lines with arrow represent the regions being targeted by the gRNAs. The vertical arrows show the positions of double strand break being introduced.

### DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Definition

As used herein, the singular forms “a”; “an” and “the” include plural references unless the context clearly dictates otherwise.

As used herein, the terms “ameliorate”, “ameliorating” and grammatical variations thereof mean to decrease the severity of the symptoms of a disease in a subject.

It is noted that in this disclosure, terms such as “comprises”, “comprised”, “comprising”, “contains”, “containing” and the like are inclusive or open-ended and do not exclude additional, un-recited elements or method steps. The terms “consists of” and “consisting of” are close ended.

A “cell”, as used herein, can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ (e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell); a cell from an endocrine system or organ (e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte)); a cell from a nervous system or organ (e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph)); a cell from a respiratory system or organ (e.g., a pneumocyte (a type I pneumocyte and a type II pneumocyte), a clara cell, a goblet cell, an alveolar macrophage); a cell from circular system or organ (e.g., myocardiocyte and pericyte); a cell from digestive system or organ (e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, a liver cell (e.g., a hepatocyte and Kupffer cell)); a cell from integumentary system or organ (e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell)), a muscle cell (e.g., myocyte), an adipocyte, a fibroblast, and a tendon cell), a cell from urinary system or organ (e.g., a podocyte, a juxtaglomerular cell, an intraglomerular mesangial cell, an extraglomerular mesangial cell, a kidney proximal tubule brush border cell, and a macula densa cell), and a cell from reproductive system or organ (e.g., a spermatozoon, a Sertoli cell, a leydig cell, an ovum, an oocyte). A cell can be normal, healthy cell; or a diseased or unhealthy cell (e.g., a cancer cell). A cell further includes a mammalian zygote or a stem cell which include an embryonic stem cell, a fetal stem cell, an induced pluripotent stem cell, and an adult stern cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and a unipotent stem cell, any of which may be induced from a somatic cell. A stem cell may also include a cancer stem cell. A mammalian cell can be a rodent cell, e.g., a mouse, rat, hamster cell. A mammalian cell can be a lagomorpha cell, e.g., a rabbit cell. A mammalian cell can also be a primate cell, e.g., a human cell.

The term “construct” or “nucleic acid construct” as used herein refers to a nucleic acid in which a polynucleotide sequence of interest is inserted into a vector, The tenn “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, and artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

The term “double-stranded” as used herein refers to one or two nucleic acid strands that have hybridized along at least a portion of their lengths. In certain embodiments, “double-stranded” does not mean that a nucleic acid must be entirely double-stranded. Instead, a double-stranded nucleic acid can have one or more single-stranded segment and one or more double-stranded segment. For example, a double-strand nucleic acid can be a double-strand. DNA, a double-strand RNA, or a double-strand DNA/RNA compound. The form of the nucleic acid can be determined using common methods in the art, such as molecular band stained with SYBR green and distinguished by electrophoresis.

The term “introduce” or “introduced” or “introducing” in the context of inserting a nucleic acid sequence into a cell, means “transfection”, or “transformation”, or “transduction” and includes reference to the incorporation of a nucleic acid sequence into a eukaryotic or prokaryotic cell wherein the nucleic acid sequence may be present in the cell transiently or may be incorporated into the genome of the cell (e.g., chromosome, plasmid, plastid, or mitochondrial DNA), converted into an autonomous replicon. The construct of the present disclosure may be introduced into a cell using any method known in the art. Various techniques for transfecting animal cells may be employed, including, for example: microinjection, retrovirus mediated gene transfer, electroporation, transfection, or the like (see, e.g., Keown et al., Methods in Enzymology (1990) 185:527-537). In one embodiment, the construct is introduced to the cell via a virus.

The term “modification” or “genetic modification” refers to a disruption at the genomic level that may result in a decrease or increase in the expression or activity of a gene expressed by a cell. Exemplary modifications can include insertion, deletions, replacement, frame shift mutations, point mutations, exon removal, etc.

“Desired modification” in the context of gene-editing refers to the genetic modification of interest, which is pursued by the manipulator. The desired modification of the present disclosure can be a modification in the genomic region that is capable of recovering, enhancing, or changing the normal function or a selected function of a gene, or increasing or decreasing the expression of a gene. “Undesired modification” is opposite to “desired modification”, which is unwanted modification resulted from random modification that is different from those are desired. In certain embodiments of the present disclosure, one or more desired modification and/or one or more undesired modification of a genomic region can be generated by CRISPR-associated system.

The term “nucleic acid” and “polynucleotide” are used interchangeably and refer to a polymeric form of nucleotides of any length, either deoxyribonucleotides or ribonucleotides, or analogs thereof, Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, a “nuclease” is an enzyme capable of cleaving the phosphodiester bonds between the nucleotide subunits of nucleic acids. A “nuclease domain” is an independently folded protein domain having nuclease activity. A “site-specific nuclease” refers to a nuclease whose functioning depends on a specific nucleotide sequence. Typically, a site-specific nuclease recognizes and binds to a specific nucleotide sequence and cuts a phosphodiester bond within the nucleotide sequence. In certain embodiments, the double-strand break is generated by site-specific cleavage using a site-specific nuclease. Examples of site-specific nucleases include, without limitation, zinc finger nucleases (ZFNs), transcriptional activator-like effector nucleases (TALENs) and CRISPR (clustered regularly interspaced short palindromic repeats)-associated (Cas) nucleases.

A site-specific nuclease typically contains a DNA-binding domain and a DNA-cleavage domain. For example, a ZFN contains a DNA binding domain that typically contains between three and six individual zinc finger repeats and a nuclease domain that consists of the FokI restriction enzyme that is responsible for the cleavage of DNA. The DNA binding domain of ZFN can recognize between 9 and 18 base pairs. In the example of a TALEN, which contains a TALE domain and a DNA cleavage domain, the TALE domain contains a repeated highly conserved 33-34 amino acid sequence with the exception of the 12^{th} and 13^{th} amino acids, whose variation shows a strong correlation with specific nucleotide recognition. For another example, Cas9, a typical Cas nuclease, is composed of an N-terminal recognition domain and two endonuclease domains (RuvC domain and HNH domain) at the C-terminus.

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

The term “subject” or “animal” or “patient” as used herein refers to human or non-human animal, including a mammal or a primate, in need of diagnosis, prognosis, amelioration, prevention and/or treatment of a disease or disorder such as viral infection or tumor. Mammalian subjects include humans, domestic animals, farm animals, and zoo, sports, or pet animals such as dogs, cats, guinea pigs, rabbits, rats, mice, horses, swine, cows, bears, and so on.

in the context of formation of a CRISPR complex, “target” refers to a guide sequence (that is, gRNA) designed to have complementarily to a genomic region (that is, a target sequence), where hybridization between the genomic region and a guide RNA promotes the formation of a CRISPR complex. The terms “complementarity” or “complementary” are used in reference to polynucleotides (i.e., a sequence of nucleotides) related by the base-pairing rules. Complementarity may be “partial,” in which only some of the nucleic acids' bases are matched according to the base pairing rules (e.g., 5, 6, 7, 8, 9, 10 out of 10 being 50%, 60%, 70%, 80%, 90%, and 100% complementary), or there may be “complete” or “total” complementarity between the nucleic acids. The degree of complementarity between nucleic acid strands has significant effects on the efficiency and strength of their hybridization to one another.

As used herein, an “effective amount” or “therapeutically effective amount” means the amount of agent that is sufficient to prevent, treat, reduce and/or ameliorate the symptoms and/or underlying causes of any disorder or disease, or the amount of an agent sufficient to produce a desired effect on a cell. In one embodiment, a “therapeutically effective amount” is an amount sufficient to reduce or eliminate a symptom of a disease. In another embodiment, a therapeutically effective amount is an amount sufficient to overcome the disease itself.

“Transcript” refers to a mRNA formed by the gene transcription for protein expression. One or more transcripts variants are formed from the same DNA segment via differential splicing. In such a process, particular exons of a gene may be included within or excluded from the messenger mRNA (mRNA), resulting in translated proteins containing different amino acids and/or possessing different biological functions.

Site-Specific Nuclease Mediated Gene Editing

Spinal muscular atrophy (SMA) is a common autosomal recessive disorder and characterized by the loss of motor neurons in the anterior horn of the spinal cord (Pearn Classification of spinal muscular atrophies, Lancet (1980) 8174, 919-922). It has been shown that the gene responsible for SMA is the Survival of Motor Neuron (SMN) gene (Lefebvre S et al., Identification and characterization of a spinal muscular atrophy-determining gene, Cell (1995) 80: 155-65), In humans, two nearly identical SMN genes (SMN1 and SMN2) exist on chromosome 5q13. Deletions or mutations within SMN1 but not the SMN2 gene cause all forms of SMA (Lefebvre S et al., Identification and characterization of a spinal muscular atrophy-determining gene, Cell (1995) 80: 155-65). SMN1 encodes a ubiquitously expressed 38 kDa SMN protein that is necessary for hnRNP assembly, an essential process for cell survival (Wan L et al., The survival of motor neurons protein determines the capacity for snRNP assembly: biochemical deficiency in spinal muscular atrophy, Mol. Cell. Biol. (2005) 25:5543-51). A nearly identical copy of the SMN1 gene, SMN2, locates near the SMN1 gene, but fails to compensate for the loss of SMN1 because the exon 7 of SMN2 is largely skipped during mRNA splicing, producing an unstable truncated protein, SMNΔ7 (Lorson C L et al., SMN oligomerization defect correlates with spinal muscular atrophy severity, Nat. Genet. (1998) 19:63-66). SMN1 and SMN2 differ by a critical C to T substitution at position 6 of exon 7 (C6U in transcript of SMN2) (Lorson C L et al., A single nucleotide in the SMN gene regulates splicing and is responsible for spinal muscular atrophy, Proc. Natl. Acad. Sci. USA (1999) 96:6307-11; Monani U R et al., A single nucleotide difference that alters splicing patterns distinguishes the SMA gene SMN1 from the copy gene SMN2, Hum. Mol. Genet. (1999) 8:1177-83). C6U does not change the coding sequence but is sufficient to cause exon 7 skipping in SMN2.

Intronic Splicing Silencer Ni (ISS-N1) was discovered as a strong inhibitory cis-element that prevents inclusion of SMN2 exon7, thus referred to as the master checkpoint of splicing regulation of SMN2 exon7 (Singh N K et al., Splicing of a critical exon of human survival motor neuron is regulated by a unique silencer element located in the last intron, Mol. Cell Biol. (2006) 26:1333-46). It has been shown that ISS-N1 is a complex regulatory element being affected by the presence of other regulatory elements upstream and downstream of ISS-N1 (Singh N N and Signh R N, Alternative splicing in spinal muscular atrophy underscores the role of an intron definition model, RNA Biol. (2011) 8:600-06; Singh N N et al., T1A1 prevents skipping of a critical exon associated with spinal muscular atrophy, Mol. Cell Biol. (2011) 31:935-54; Singh N N et al., An intronic structure enabled by a long-distance interaction serves as a novel target for splicing correction in spinal muscular atrophy, Nucl. Acids Res. (2013) 41:8144-65; Singh N N et al., Modulating role of RNA structure in alternative splicing of a critical exon in the spinal muscular atrophy genes, Nucl. Acids Res., (2007)35:371-89), Antisense oligonucleotides have been successfully designed to anneal to complementary ISS-N1 in the pre-mRNA to redirect SMN2 splicing and include exon7 (Finkel R S et al., Nusinersen versus sham control in infantile-onset spinal muscular atrophy, N Engl J Med (2017) 377:1723-32).

Therefore, the present disclosure in one aspect provides compositions and methods of gene editing using site-specific nuclease to disrupt the ISS-N1 region of the SMN2 gene in a cell, thus increasing inclusion of exon 7 and expression of SMN protein in the cell. In some embodiments, the site-specific nuclease includes a CRISPR-associated (Cas) nuclease, a zinc finger nuclease (ZFN) or a transcriptional activator-like effector nuclease (TALEN).

In one embodiment, the method comprises introducing a composition to a human cell, wherein the composition comprises a site-specific nuclease targeting the ISS-N1 region of human SMN2 gene. When the composition is introduced to the cell, a double strand break is generated in the ISS-N1 region, which leads to an indel at the ISS-N1 region via NHEJ repair pathway. The indel disrupts the function of the ISS-N1, altering splicing, increasing inclusion of exon 7 in the SMN2 mRNA and increasing SMN protein in the cell.

In one embodiment, the method comprises introducing a composition to a human cell, wherein the composition comprises: (1) a Cas nuclease or a nucleic acid encoding the same, and (2) a gRNA or a nucleic acid encoding the same, wherein the gRNA targets the ISS-N1 region of human SMN2 gene.

The term “CRISPR (RNA-guided clustered regularly interspaced short palindromic repeats)/Cas system” originally refers to transcripts and other elements in the prokaryotic cells involved in the expression of or directing the activity of CRISPR-associated (“Cas”) genes, including sequences encoding a Cas nuclease that cleaves the nucleic acid sequence and generates double strand break (DSB), a guide sequence, a trans-activating CRISPR (tracr) sequence, a tracr-mate sequence, or other sequences and transcripts from a CRISPR locus. In eukaryotic cells, the CRISPR/Cas system comprises a CRISPR-associated nuclease and a small guide RNA. The target DNA sequence (the protospacer) contains a “protospacer-adjacent motif” (PAM), a short DNA sequence recognized by the particular Cas protein being used. In certain embodiments, the CRISPR. system comprises CRISPR/Cas system of type I, type II, and type III, which comprises protein Cas3, Cas9 and Cas10, respectively.

The RNA-guided endonuclease Cas9 is a component of the type II CRISPR system widely utilized generate gene-specific knockouts in a variety of model systems. In one embodiment of the present disclosure, the CRISPR/Cas nuclease is a “sequence-specific nuclease”. Introduction of ectopic expression of Cas9 and a single guide RNA (gRNA) is sufficient to lead to the formation of double-strand breaks (DSBs) at a specific genomic region of interest, which leads to an indel via MEI pathway. Indels often result in frameshift mutations, except when the number of inserted/deleted nucleotides is a multiple of 3.

Along with Cas endonuclease, CRISPR experiments require the introduction of a guide RNA containing an approximately 15 to 30 base sequence specific to a target nucleic acid (e.g., DNA). A gRNA designed to target a genomic region of interest, for example, a particular exon encoding a functional domain of a protein, will generate a mutation in each gene that encodes the protein. The resulted modified genomic region may comprise one or more variants, each of which is different in the mutation. For example, the mutation will result in a modified genomic region with a desired modification, and/or a modified genomic region with an undesired modification. This approach has been widely utilized to generate gene-specific knockouts in a variety of model systems. In certain embodiments, a gRNA has a length of 15, 16, 17, 18, 19, 20, 21, 22, 23, 24, 25, 26, 27, 28, 29 or 30 nucleotides, gRNA can be delivered into a eukaryotic cell or a prokaryotic cell as RNA or by transfection with a vector (e.g., plasmid) having a gRNA-coding sequence operably linked to a promoter.

In certain embodiments, the Cas nuclease and the gRNA. are derived from the same species. In certain embodiments, the Cas nuclease is derived from, for example, Staphylococcus aureus, Staphylococcus epidermidis, Staphylococcus sciuri, Pseudomonas aeruginosa, Enterococcus faecium, Enterococcus faecalis, Escherichia coli, Klebsiella pneumoniae, Streptococcus pneumoniae, Streptococcus pyrogenes, Lactobacillus bulgaricus, Streptococcus thermophilusVibrio cholera, Achromobacter xylosoxidans, Burkholderia cepacia, Citrobacter diversus, Citrobacter freundii, Micrococcus leuteus, Proteus mirabilis, Proteus vulgaris, Staphylococcus lugdunegis, Salmonella typhi, Streptococcus Group A, Streptococcus Group B, S. marcescens, Enterobacter cloacae, Bacillus anthracis, Bordetella pertussis, Clostridium sp., Clostridium botulinum, Clostridium tetani, Corynebacterium diphtheria, Moraxalla (Brauhatnella) catarrhalis, Shigella spp., Haemophilus aemophilus influenza, Stenotrophomonas maltophili, Pseudomonas perolens, Pseuomonas fragi, Bacteroides fragilis, Fusobacterium, sp. Veillonella sp., Yersinia pestis, and Yersinia pseudotuberculosis.

A gRNA can be designed using any known software in the art, such as Target Finder, E-CRISPR, CasFinder, and CRISPR Optimal Target Finder.

In certain embodiments, the composition described herein comprises a nucleic acid encoding the CAS nuclease or the gRNA, wherein the nucleic acid is contained in a vector. In some embodiments, the composition comprises CAS nuclease protein and a DNA encoding the gRNA. In some embodiments, the composition comprises a first nucleic acid encoding the CAS nuclease and a second nucleic acid encoding the gRNA, whereas the first and the second nucleic acids are contained in one vector. In some embodiment, the first and the second nucleic acids are contained in two separate vectors. In some embodiments, at least one vector is a viral vector.

In certain embodiments, the ISS-N1 region comprises SEQ ID NO: 1 (CCAGCATTATGAAAG), In some embodiments, the ISS-N1 region consists of SEQ ID NO: 1.

In some embodiments, the composition includes two gRNAs targeting the sequences flanking the ISS-N1 region. When the composition is introduced into the cell, the two gRNAs in combination of Cas nuclease delete the ISS-N1 region, disrupting the inhibitory function of ISS-N1.

In some embodiments, the composition, when being introduced into the cell, generates a modified ISS-N1 in the cell which does not substantially disrupt the splicing inhibitory function of ISS-N1. In such case, a second gRNA that targets the modified ISS-N1 can be introduced to the cell to further edit the ISS-N1 region, generating altered ISS-N1 region that confers increased inclusion of exon7 in SMN2 mRNA.

Therefore, in certain embodiments of the methods described herein, the composition includes a first gRNA and a second gRNA (or two constructs or DNA fragments for transcribing the first gRNA and the second gRNA, respectively). The first gRNA targets the ISS-N1 region of the SMN2 gene in a plurality of human cells and generates a number of variants of modified ISS-N1 region comprising desired (with splicing inhibitory function of ISS-N1 disrupted) and undesired modifications (with splicing inhibitory function of ISS-N1 not substantially disrupted) in different subset of cells, respectively. The second gRNA is designed to target the undesired modification in order to further generate the desired modification and enhance the gene-editing efficiency. In some embodiments, a plurality of undesired modifications exists. In such case, a plurality of gRNA targeting the undesired modifications may be used. For example, in certain embodiments, the present disclosure comprises a third gRNA that targets an undesired mutation different from the one targeted by the second gRNA to further enhance the gene-editing efficiency.

In certain embodiments, the genetic material for expressing the CRISPR/Cas nucleases is specifically composed of a construct or DNA fragment for transcribing a sequence containing the first gRNA and the second gRNA (or two constructs or DNA fragments for transcribing the first gRNA and the second gRNA, respectively) and for expressing Cas protein; or is specifically composed of a construct or DNA fragment for transcribing the sequence containing the first gRNA and the second gRNA (or two constructs or DNA fragments for transcribing the first gRNA and the second gRNA, respectively) and a construct or DNA fragment or RNA for expressing Cas protein; or is specifically composed of a RNA sequence containing the first gRNA and the second gRNA (or the first gRNA and the second gRNA) and a construct or DNA fragment or RNA for expressing Cas protein. In sonic preferred embodiments, the first and second gRNAs are constructed within the same vector, so that to increase the chance for the second gRNA to target the undesired mutation. In some embodiments, the Cas protein is introduced into the cells as a polypeptide. In some embodiments, the gRNAs are introduced into the cells without using DNA construct, i.e. as RNAs.

In certain embodiments, the first gRNA and the second gRNA contained in the same construct are polycistronic gRNA, such that both gRNAs are located within the same polycistronic operon and transcribed in the same time. In certain embodiments, the first gRNA and the second gRNA contained in the same construct are located in two poly- or mono-cistronic operons each with its own promoter and is transcribed individually.

In certain embodiments, said gRNA is an RNA with a palindromic structure which is formed by partial base-pairing between crRNA and tracrRNA; said crRNA contains an RNA fragment capable of complementarily binding to the target site.

In certain embodiments, the undesired modification of the genomic region is an insertion, a deletion, ora replacement of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides. The sequences of the resulted modified genomic region generated by the first gRNA can be determined by genotyping using conventional means in the art, for example, by next generation sequencing (NGS).

The methods conceived and disclosed herein also include using variants or engineered site-specific nuclease that can modify the target sequence without causing double strand break or using NHEI or homologous recombination. For example, David Liu's group at MIT has described dCas9, an engineered Cas9 that fuses Cas9 and a cytidine deaminase enzyme, which does not induce dsDNA breads but mediates a C to T (or G to A) substitution (See Komor A C et al., Programmable editing of a target base in genomic DNA without double-stranded DNA cleavage, Nature (2016) 19:533:420-24). A fusion enzyme having adenine deaminase activity has also been developed (Gaudelli M N et al., Programmable base editing of A·T to G·C in genomic DNA without DNA cleavage, Nature (2017) 551: 464-71). In some embodiments, the gene-editing methods include introducing such engineered site-specific enzymes, e.g., fusion of dCas9, ZFN or TALEN with a cytidine deaminase, to a cell, thereby editing the ISS-N1 region to disrupt the function of ISS-N1. In one embodiment, the gene-editing methods include introducing an engineered site-specific enzyme to a cell to mediate a T to C substitution at the position 6 of exon 7 of SMN2 gene, converting the SMN2 gene to SMN1 aerie.

Treatment of Spinal Muscular Atrophy

In another aspect, the present disclosure provides a method for treating or ameliorating spinal muscular atrophy (SMA). In one embodiment, the method comprises administering a therapeutically effective amount of the composition described herein to a subject having at least one symptom associated with spinal muscular atrophy (SMA).

SMA is a genetic disorder characterized by degeneration of spinal motor neurons. SMA is caused by the homozygous loss of both functional copies of the SMN1 gene. However, in humans, the SMN2 gene has the potential to code for the same protein as SMN1 and thus overcome the genetic detect of SMA patients. SMN2 contains a translationally silent mutation (C→T) at position +6 of exon 7, which results in inefficient inclusion of exon 7 in SMN2 transcripts. Therefore, the predominant form of SMN2 protein, encoded by SMN2 transcripts lacking exon 7, is unstable and inactive. Thus, therapeutic composition capable of modulating SMN2 splicing such that the percentage of SMN2 transcripts containing exon 7 is increased, would be useful for the treatment of SMA.

In certain embodiments, the subject being administered with the composition described herein has one or more indicator of SMA. In certain embodiments, the subject has reduced electrical activity of one or more muscles. In certain embodiments, the subject has a mutant SMN1 gene. In certain embodiment, the subject's SMN1 gene is absent or incapable of producing functional SMN protein. In certain embodiments, the subject is diagnosed by a genetic test. In certain embodiments, the subject is identified by muscle biopsy. In certain embodiments, the subject is unable to sit upright. In certain embodiments, the subject is unable to stand or walk. In certain embodiments, the subject requires assistance to breathe and/or eat. In certain embodiment, the subject is identified by electrophysiological measurement of muscle and/or muscle biopsy.

In certain embodiments, the subject has SMA type I. In certain embodiments, the subject has SMA type II. In certain embodiments, the subject has SMA type III. In certain embodiments, the subject is diagnosed as having SMA in utero. In certain embodiments of the method described herein, the subject is an infant. In certain embodiments, the subject is diagnosed as having SMA within one week after birth. In certain embodiments, the subject is diagnosed as having SMA within one month of birth. In certain embodiments, the subject is diagnosed as having SMA by 3 months of age. In certain embodiments, the subject is diagnosed as having SMA by 6 months of age. In certain embodiments, the subject is diagnosed as having SMA by 1 year of age. in certain embodiments, the subject is diagnosed as having SMA between 1 and 2 years of age, In certain embodiments, the subject is diagnosed as having SMA between 1 and 15 years of age. In certain embodiments, the subject is diagnosed as having SMA when the subject is older than 15 years of age.

The composition described herein can be administered to the subject using any route or method known in the art. In some embodiments, the composition is administered systematically to the subject. In certain embodiments, the composition is administrated through subcutaneous injection. In certain embodiments, the composition is administrated through intravenous injection.

In certain embodiments of the method described herein, the composition is administered into the central nervous system of the subject. in certain embodiments, the composition is administered into the cerebrospinal fluid (CSF) of the subject. In certain embodiments, the composition is administered into the intrathecal space of the subject. In certain embodiments, the composition is administered via intramuscular injection.

CSF is a clear fluid that fills the ventricles, is present in the subarachnoid space, and surrounds the brain and spinal cord. CSF is produced by the choroid plexuses and via the weeping or transmission of tissue fluid by the brain into the ventricles. The choroid plexus is a structure lining the floor of the lateral ventricle and the roof of the third and fourth ventricles. Certain studies have indicated that these structures are capable of producing 400-600 ccs of fluid per day consistent with an amount to fill the central nervous system spaces four times in a day. In adult humans, the volume of this fluid has been calculated to be from 125 to 150 ml (4-5 oz). The CSF is in continuous formation, circulation and absorption. Certain studies have indicated that approximately 430 to 450 ml (nearly 2 cups) of CSF may be produced every day. Certain calculations estimate that production equals approximately 0.35 ml per minute in adults and 0.15 per minute in infant humans. The choroid plexuses of the lateral ventricles produce the majority of CSF. It flows through the foramina of Monro into the third ventricle where it is added to by production from the third ventricle and continues down through the aqueduct of Sylvius to the fourth ventricle. The fourth ventricle adds more CSF, the fluid then travels into the subarachnoid space through the foramina of Magendie and Luschka. It then circulates throughout the base of the brain, down around the spinal cord and upward over the cerebral hemispheres. The CSF empties into the blood via the arachnoid villi and intracranial vascular sinuses.

In certain embodiments, the method described herein ameliorates at least one symptom of SMA in the subject. In some embodiments, the composition administered to the subject enters neurons, e.g., motor neurons, generating a double strand break in the ISS-N1 region of the SMN2 gene in the neuron, which leads to an indel at the ISS-N1 region via NHEJ repair pathway. The indel disrupts the splicing inhibitory function of the ISS-N1, increasing inclusion of exon 7 in the SMN2 mRNA and increasing SMN protein in the cell.

In certain embodiments, improved SMN function in non-neuronal cells provides improved neuronal cell function, whether or not SMN function inside neurons is improved. For example, in certain embodiments, systemic administration of pharmaceutical compositions of the present invention results in increased SMN protein in muscle cells. Such increased SMN protein in muscle cells may provide a benefit to the motor-neurons associated with that muscle cell or to neurons generally. in such embodiments, the muscle cell having restored SMN function may provide a factor that improves neuronal viability and/or function.

### EXAMPLES

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1

The following example illustrates an exemplary composition and method for increasing SMN2 expression in a cell.

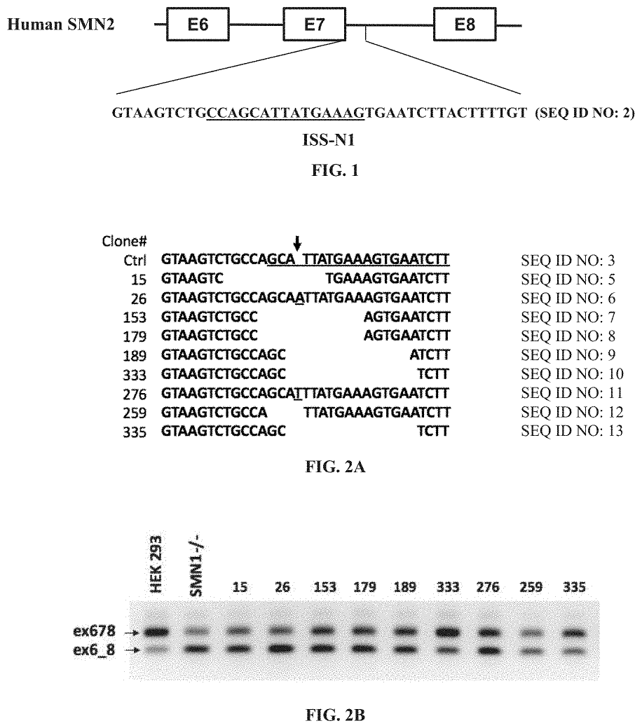

SMN1 knock-out cell line (SMN1 −/−) was generate based on HEK293 cells. spCAS9/gRNA RNP complex was used for transfection. The gRNA targeted sequence was underlined and the CAS9 cutting site was indicated by an arrow head in the wild type sequence (Ctrl). Single cell clones were generated and genotyped by sequencing. The genomic sequence for each homozygous clone were shown in FIG. 2A. RT-PCR was performed with mRNA from each clone with primers SMNex6-F TTCTCTTGATGATGCTGATGCTTTGG (SEQ ID NO: 19) and Exon8 Primer R GTCTGATCGTTTCTTTAGTGGTGTCA (SEQ ID NO: 20). As shown in FIG. 2B, the top band indicated the splicing form includes exon7 (exon 678) and the bottom band indicates the splicing form with exon7 skipping (ex6_8)

As shown in FIG. 2B, all clones (in particular, clones 153, 179, 189, 333, 276 and 335) had increased splicing form exon 678, leading to increased expression of functional and stable SMN2 protein.

### Example 2

The following example illustrates an exemplary composition and method for increasing SMN2 protein in a cell.

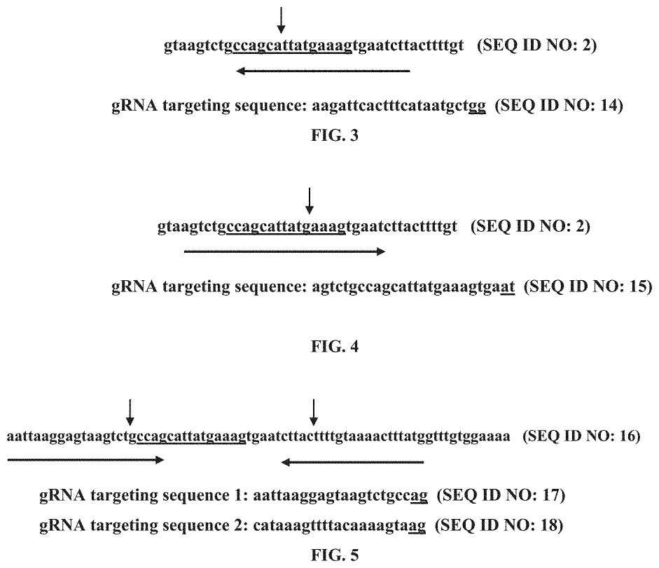

As illustrated in FIG. 3, a gRNA is designed to target the ISS-N1 with spCas9. The ISS-N1 region is underlined. The gRNA targeted region is shown with a horizontal line with arrow, and the CAS9 cutting site is indicated by a vertical arrow head. The nucleic acid encoding the gRNA is cloned into a vector including the nucleotide encoding spCas9. The construct is then introduced into HEK293 cells with SMN2 minigene. The results indicate that the inclusion of exon 7 in the mRNA of SMN2 increases.

### Example 3

The following example illustrates an exemplary composition and method for increasing SMN2. protein in a cell.

As illustrated in FIG. 4, a gRNA is designed to target the ISS-N1 with saCas9. The ISS-N1 region is underlined. The gRNA targeted region is shown with a horizontal line with arrow, and the CAS9 cutting site is indicated by a vertical arrow head. The nucleic acid encoding the gRNA is cloned into a vector including the nucleotide encoding saCas9. The construct is then introduced into HEK293 cells with SMN2 minigene. The results indicate that the inclusion of exon 7 in the mRNA of SMN2 increases.

### Example 4

The following example illustrates an exemplary composition and method for increasing SMN2 protein in a cell.

As illustrated in FIG. 5, two gRNAs are designed to target the ISS-N1 with xCas9-3.7 (Hu J H et al., Evolved Cas9 variants with broad PAM compatibility and high DNA specificity, Nature (2018) 556:57-63). The ISS-N1 region is underlined. The gRNA targeted regions are shown with horizontal lines with arrow, and the CAS9 cutting sites are indicated by vertical arrow heads. The nucleic acid encoding the gRNAs are cloned into a vector includes the nucleotide encoding xCas9-3.7. The construct is then introduced into HEK293 cells with SMN2 minigene. The results indicate that the double strand breaks introduced by the gRNAs leads to the deletion of the ISS-N1 region and the inclusion of exon 7 in the mRNA of SMN2 increases.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. A composition comprising
(1) a CRISPR-associated (Cas) nuclease or a nucleic acid encoding the same; and
(2.) a gRNA or a nucleic acid encoding the same, wherein the gRNA targets the ISS-N1 region of human SMN2 gene.

2. The composition of claim 1, wherein the ISS-N1 region comprises SEQ ID NO: 1.

3. The composition of claim 1, which comprises a nucleic acid encoding the Cas nuclease or the gRNA, wherein the nucleic acid is contained in a viral vector.

4. The composition of claim 1, which is capable of enhancing expression of SMN protein in a cell when the composition is introduced into the cell.

5. The composition of claim 4, wherein inclusion of exon 7 of SMN2 mRNA in the cell is increased.

6. The composition of claim 1, which is capable of ameliorating at least one symptom of SMA when administered into a subject having SMA.

7. The composition of claim 6, wherein inclusion of exon 7 of SMN2 mRNA in a motor neuron in the subject is increased.

8. The composition of claim 1 which is capable of generating a modified ISS-N1 in a motor neuron of a subject having SMA when administered into the subject.

9. The composition of claim 1, further comprises a second gRNA, wherein the first and the second gRNA. are capable of deleting a nucleotide sequence in the ISS-N1 region in a cell when the first and second gRNA are introduced in the cell.

10. The composition of claims 1, which is capable of generating a modified ISS-N1 in a cell when the composition is introduced to the cell, wherein the composition further comprises a second g NA or a nucleic acid encoding the same, wherein the second gRNA targets the modified ISS-N1.

11. A method of enhancing expression of SMN protein in a human cell, the method comprising introducing a composition to the human cell, said composition comprising:
(1) a CRISPR-associated (Cas) nuclease or a nucleic acid encoding the same, and
(2) a gRNA or a nucleic acid encoding the same, wherein the gRNA targets the ISS-N1 region of human SMN2 gene.

12. The method of claim 11 wherein the composition comprises a nucleic acid encoding the Cas nuclease or the gRNA, and wherein the nucleic acid is contained in a viral vector.

13. The method of claim 11, wherein the cell is a motor neuron.

14. The method of claim 11, wherein inclusion of exon 7 of SMN2 mRNA in the cell is increased.

15. The method of claims 11, which generates a modified. ISS-N1 in the cell, wherein the composition further comprises a second gRNA or a nucleic acid encoding the same, wherein the second gRNA targets the modified ISS-N1.

16. A method comprising administering a composition to a subject having at least one symptom associated with spinal muscular atrophy (SMA), said composition comprising:
(1) a CRISPR-associated (Cas) nuclease or a nucleic acid encoding the same, and
(2) a gRNA or a nucleic acid encoding the same, wherein the gRNA targets the ISS-N1 region of human SMN2 gene.

17. The method of claim 16, wherein the subject is an infant.

18. The method of claim 16 wherein the composition comprises a nucleic acid encoding the CAS nuclease or the gRNA, and wherein the nucleic acid is contained in a viral vector.

19. The method of claim 16, the composition is administered systematically to th subject.

20. The method of claim 19, wherein the composition is administered through subcutaneous injection.

21. The method of claim 19, wherein the composition is administrated through intravenous injection.

22. The method of claim 16, wherein the composition is administered into the central nervous system of the subject.

23. The method of claim 16, wherein the composition is administered into the cerebrospinal fluid of the subject.

24. The method of claim 16, wherein the composition is administered into the intrathecal space of the subject.

25. The method of claim 16, wherein the composition is administered via intramuscular injection.

26. The of claim 16, which ameliorates at least one symptom of SMA in the subject.

27. The method of claim 16, wherein inclusion of exon 7 of SMN2 mRNA in a motor neuron in the subject is increased.

28. The method of claim 16, generates a modified ISS-N1 in a motor neuron of th subject.

29. The method of claims 28, wherein the composition further comprises a second gRNA or a nucleic acid encoding the same, wherein the second gRNA targets the modified ISS-N1.
